(19) United States
(12) Patent Application Publication
(10) Pub. No.: US 2008/0004666 A1
(43) Pub. Date: Jan. 3, 2008

(54) Device for Hemodynamic Stabilization During Tachycardias

(76) Inventor: Patrick Schauerte, Aachen (DE)

Correspondence Address: PACESETTER, INC., 15900 VALLEY VIEW COURT, SYLMAR, CA, 91392-9221, US

(21) Appl. No.: 11/578,442

(22) PCT Filed: Apr. 12, 2005
(86) PCT No.: PCT/EP05/03832

§ 371 Date: Jul. 24, 2007

(30) Foreign Application Priority Data
Apr. 14, 2004 (GB) 0408335.8

(51) Int. Cl. A61N 1/365

(52) U.S. Cl. 607/14

(57) ABSTRACT

Implantable device, in particular implantable cardioverter-defibrillator, for responding to tachycardia events in a patient's heart comprising a control device, a first interface connected to said control device for receiving first signals representative of tachycardia events and connectable to a first sensor device for detecting tachycardia events, a second interface connected to said control device and connectable to a first stimulation electrode, and said control device being arranged for providing at least one stimulation pulse to said second interface in response to at least one of said first signals received at said second interface for responding to tachycardia events, wherein said control device is arranged for providing at least one first stimulation pulse to said second interface upon continued presence of said first signals at said first interface for at least intermittently improving the cardiac output during continued tachycardia events.

### BACKGROUND OF THE INVENTION

The present invention relates to hemodynamic stabilization during tachycardias.

Sudden cardiac death is the single most reason for death in humans and accounts for about 20% of all deaths in man. Besides bradycardic arrhythmias ventricular tachycardias (VT) and ventricular fibrillation (VF) constitute the vast majority of cases of sudden cardiac death. In most cases, VT is the prevailing arrhythmia which accelerates to VF due to the ischemia and arterial hypotension associated with prolonged episodes of VT.

The implantable cardioverter-defibrillator (ICD) which can terminate VT/VF and has been proven to significantly prolong life in patients after survived VT/VF (secondary prophylaxis) or in patients at high risk to experience VT/VF (primary prophylaxis).

In case of a ventricular tachycardia, an ICD basically has 2 treatment algorithms. According to the device settings which can be programmed by the physician the device selects to initiate an overdrive stimulation (=antitachycardic pacing: ATP) or cardioversion/defibrillation (CV) shock. ATP is generally preferred as it allows to terminate VT without painful CV shocks thereby increasing patients' quality of life (QOL) and preserving ICD battery longevity. During ATP the ventricular pacing lead delivers short trains of ventricular electrical stimuli with a slightly shorter cycle length than the VT cycle length. Typically more than one ATP attempt has to be delivered by the device and often several ATP attempts have to be delivered until VT terminates.

Ongoing VT is usually accompanied by severe arterial hypotension due to the changed contraction pattern and tachycardic heart rate. In general, the higher the VT rate and the longer the VT continues the lower cardiac output and arterial pressure will be. In fact as ATP is delivered at even higher rates than VT the blood pressure during ATP may decline even more. Therefore, prolonged ATP will cause arterial hypotension and acute heart failure finally causing patient's syncope with consecutive physical damage. This has led all ICD manufacturers to limit the maximal number of ATP attempts.

If ATP fails or is not programmed the device will deliver a CV shock. Depending on the chosen CV energy and the age of the battery it takes 4-20 seconds to charge the capacitor During the charging period VT continues. As the VT already lasted for several seconds in the detection period (period in which the ICD detects a sustained arrhythmia) and/or numerous failed ATP attempts had been undertaken before the patient's hemodynamic situation progressively worsens and many patients loose their consciousness and fall (syncope). Although syncope prevents that the patient feels the painful CV/defibrillation shock this loss of consciousness is generally not desirable as the patients may get hurt depending on the location and activity during spontaneous VT (e.g. during traffic, walking).

Besides VTs that can be terminated by ATP or CV there are situations in which VT is ongoing or rapidly recurring despite aggressive ATP attempts or repeated cardioversion shocks. These most worrisome VTs are incessant VTs or VTs clustering as electrical storm (see D1 below). Incessant VT which is defined as a ventricular tachycardia that either cannot be terminated by CV or immediately recurs after CV. In fact a history of electrical storm or incessant VT is a contra-indication for implanting an ICD (see D2). This is because the ICD typically will deliver multiple consecutive shocks either because the VT is not terminated by the CV shocks or because the VT quickly recurs within seconds or minutes after initial successful CV. This leads to substantial stress of the patient and early depletion of the ICD battery. If the patient reaches medical aid, treatment options are also limited and treatment algorithms include i.v. beta-blockade, sedation and amiodarone infusion (see D1, D3). However, many of the patients already are on chronic beta-blockade and amiodarone therapy because of a history of significant CAD and previous VT. In addition, there is a latency to the onset of an anti-arrhythmic effect of amiodarone in patients with incessant VT (see D4, D5). Catheter ablation of the VT constitutes the ultimo ratio in some of these patients but is confined to highly specialized centers (see D6, D7). For all these reasons during electrical storm or incessant VT the physician faces the uncomfortable situation in which he knows that the traditional amatory for tachycardia treatment does not work any longer.

List of Cited Literature:

D1 Nademanee K et al. Circulation. 2000;102:742-7;

D2 Gregoratos G et al. J Am Coll Cardiol. 2002;40:1703-19;

D3 John C et al. Am J Cardiol 2002;90:853-859;

D4 Kowey P R et al. Circulation 1995;92;3255-3263;

D5 Scheinemann M et al. Circulation 1995;92;3264-3272;

D6 Trappe H. J. et al. Z Kardiol 1991;80:720-726;

D7 Bänsch et al. Circulation 2003;3011-3016.

### SUMMARY OF THE INVENTION

It is thus an object of the present invention to provide a cardiac stimulator capable of augmenting cardiac performance in patients with ongoing ventricular tachycardia until other means to terminate the arrhythmia like ATP, cardioversion/defibrillation or drug therapy are coming to an effect.

The above object is achieved by an implantable device showing the features of claim 1.

In a first aspect of the present invention the above object is achieved by a method and a device for providing critically timed ventricular stimuli to the heart during VT which do not terminate the arrhythmia but intermittently suppress the breakthrough of the VT and improves myocardial function by a postextrasystolic potentiation mechanism.

In another aspect of the present invention the cardiac excitation pathway during VT is modified by introduction of stimulated ventricular beats to achieve ventricular fused beats with shorter QRS complexes thereby improving the ventricular contraction pattern during VT.

In a further aspect of the present invention atrioventricular synchronization is achieved at a heart rate above the rate during VT by continuous atrial stimulation above the spontaneous VT rate.

Further embodiments of the present invention will become apparent from the dependent claims and the following description of preferred embodiments which refers to the appended figures.

### BRIEF DESCRIPTION OF THE DRAWINGS

FIG. 1 is an illustration of an embodiment of the stimulation algorithm according to the present invention with paired ventricular stimulation (PVS). Panel A: spontaneous VT with a tachycardia cycle length of 406 ms. Panel B: at a coupling interval of 280 ms, a single ventricular premature beat (VPB) from the catheter within the right ventricular apex was triggered to a spontaneous VT beat. This interval was short enough to excite the ventricle during VT but too short to allow for an adequate filling of the left ventricle (LV). By the time, the next VT beat would be expected the ventricle has already been partially or fully depolarized by the extrasystole (note the presence of fusion beats, which differ from the superior axis, LBBB observed during RVA pacing during SR). This results in a prolongation of the post-pacing interval (503 ms) which is longer than the VT Cycle length. The successive beat is again a spontaneous VT beat.

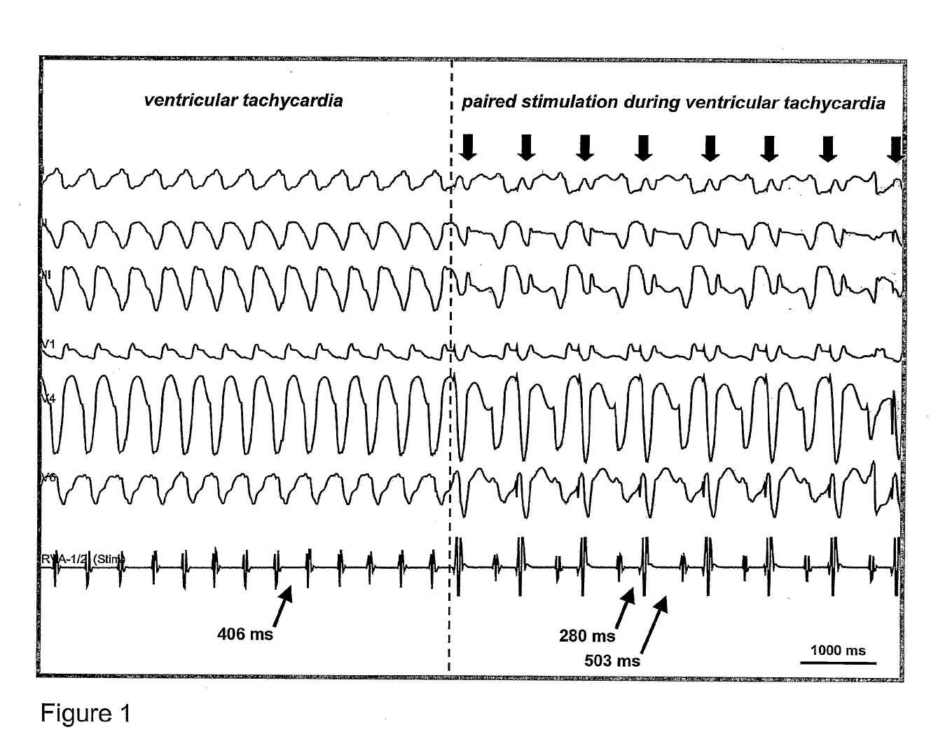

FIG. 2 is an illustration of the hemodynamic effect of paired ventricular stimulation (PVS) in a patient with ventricular tachycardia and severely depressed left ventricular function (EF: 19%). The VT cycle length was 450 ms. Surface ECG lead II and an arterial blood pressure tracing are depicted. The numbers within the pressure tracings indicate systolic (upper value) diastolic (bottom value) and mean arterial pressure (underlined value) of a given arterial pressure wave. The dotted line denotes the initiation of 1:1 paired ventricular stimulation. Paired stimuli were triggered to the VT beat at a coupling interval of 240 ms (*). The coupled beat did not produce a significant pressure wave but prevented the breakthrough of the next VT reentrant beat and consecutively increased the length of the diastolic filling period. During paired stimulation the number of arterial pressure waves was reduced by half. Of note: it took 5-6 paired stimuli until the pressure values peaked and reached a plateau during paired stimulation.

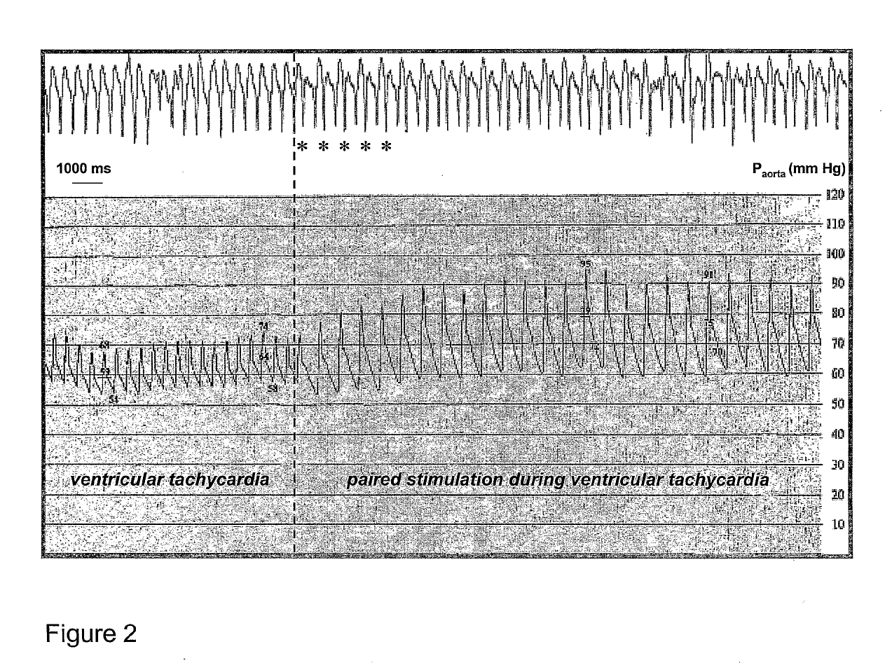

FIG. 3 is an illustration of the hemodynamic effects during the on- and offset of paired stimulation (PVS). Abbreviations as in FIG. 1. The augmentation of arterial pressure persisted for 2 VT beats after cessation of PVS until it declined to a new steady state value within the next 3-4 VT beats.

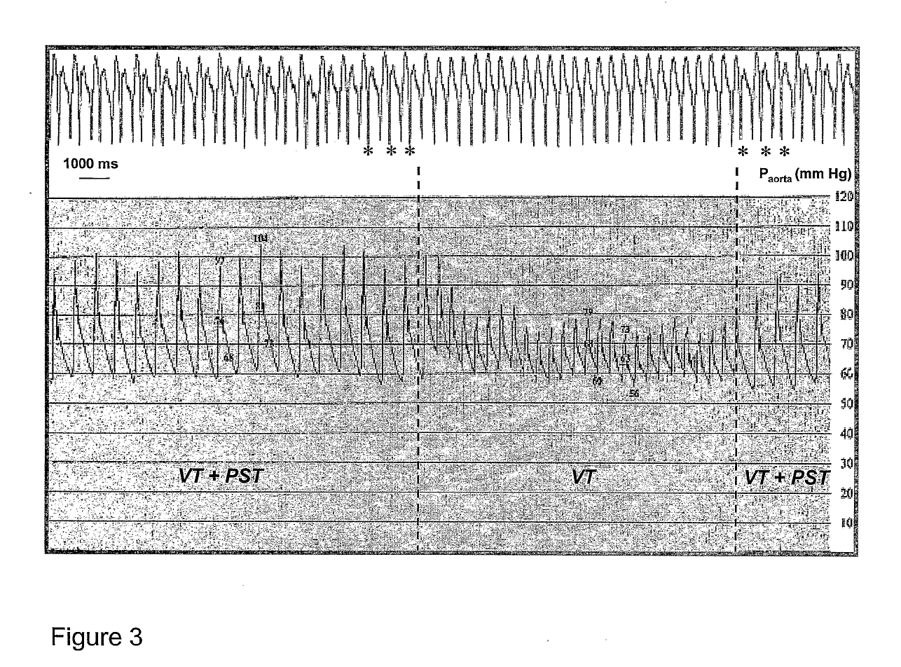

FIG. 4 is an illustration of the hemodynamic response to paired stimulation in a patient with severely reduced LV function (EF: 30%) and a VT cycle length of 320 ms. A significant increase of mean arterial pressure was observed during paired stimulation with a coupling interval of 200 ms. Periodic changes of the arterial pressure occurring at low frequencies are due to respiratory modulations of cardiac preload. Abbreviations as in FIG. 1.

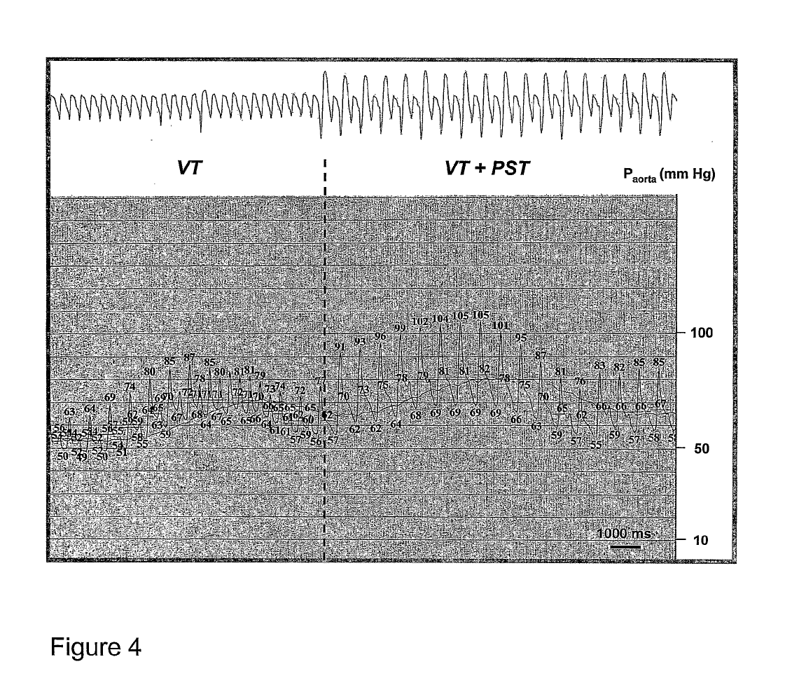

FIG. 5 is an illustration of the hemodynamic effects of paired stimulation in 14 patients.

FIG. 6 is an illustration of intermittent paired stimulation for hemodynamic augmentation to allow prolonged antitachycardic stimulation attempts during ongoing tachycardia.

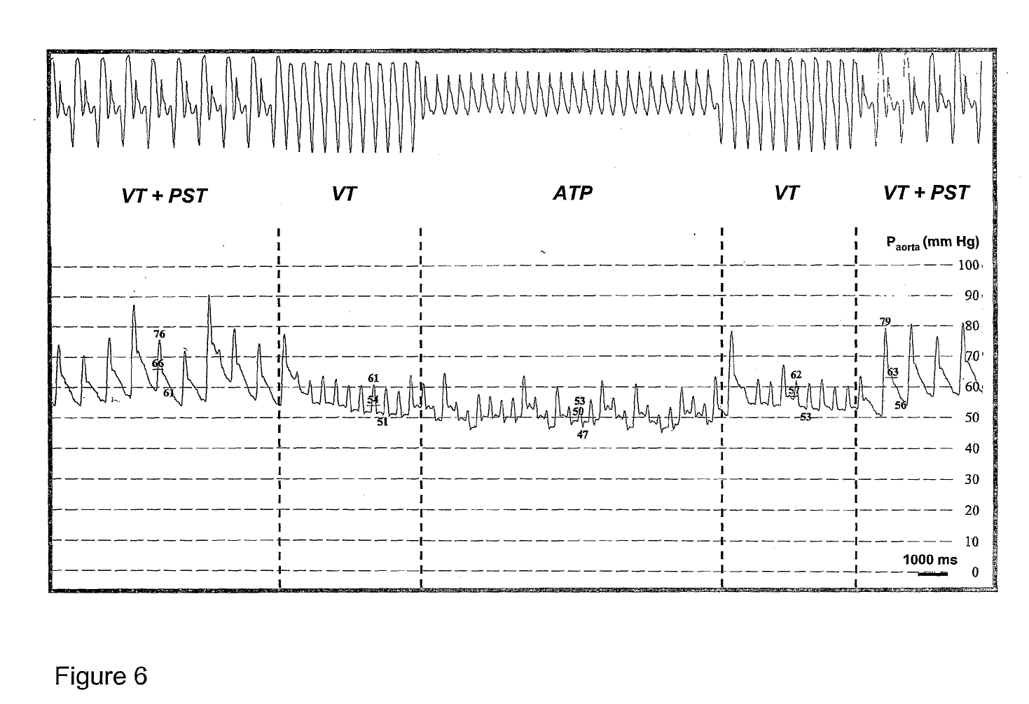

FIG. 7 is a schematic illustration of preferred embodiment of an implantable device 1 according to the present invention.

### DETAILED DESCRIPTION OF THE INVENTION

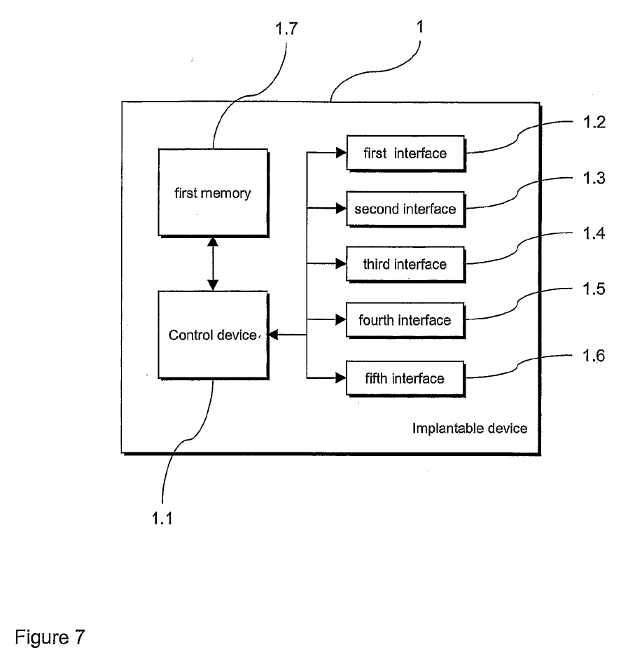

In the following, a preferred embodiment of an implantable device 1 according to the present invention will be first described with reference to FIG. 7. The implantable device 1 is an implantable cardioverter-defibrillator (ICD) with a control device 1.1 connected to a first interface 1.2, a second interface 1.3, a third interface 1.4, a fourth interface 1.5, a fifth interface 1.6 and a first memory 1.7.

A ventricular extrasystole which occurs shortly outside the effective ventricular refractory period generates a postextrasystolic pause and leads to an augmentation of the arterial pressure wave initiated by the next spontaneous beat. This phenomenon is easily recognized in the arterial pressure recording during left heart catheterization and has originally been described by Langendorff (see D6).

The present invention provides a device for hemodynamic stabilization of VT during ongoing VT by introducing paced ventricular premature beats (VPBs) during spontaneous VT in a paired ventricular stimulation (PVS) mode of said control device 1.1. The stimulated beats will be coupled to each, single or multiple ventricular tachycardia beats and will be introduced briefly after termination of the ventricular refractory period. The short first coupling interval (CI-1) is not sufficient to allow for an adequate diastolic filling of the heart resulting either in no or only small amplitude arterial pressure wave. Due to the interposed extrasystole the next VT beat cannot electrically break through because the time interval between the paced extrasystole and the electrical exit of the subsequent local reentrant circuit of the VT beat is shorter than the ventricular refractory period during VT. This will result in a postextrasystolic pause which is longer than the spontaneous VT cycle length. Consequently the diastolic filling period of the heart is prolonged leading to an augmentation of the amplitude of the first pressure wave after the paced extrasystole. This pressure wave is generated by the next spontaneous VT beat. Besides prolongation of the diastolic filling period and prevention of the breakthrough of spontaneous VT beats mechanisms like post-extrasystolic potentiation (see Cooper et al. Circulation 1993;88:2962-2971) are operative during coupled stimulation. Because the effect of postextrasystolic potentiation decays within 2-5 beats after a single S-1, coupled beats may only be introduced every 2^{nd}, 3^{rd} or n^{th} spontaneous VT beat with n being an integer greater than 1.

Although short-term experiences with coupled stimulation during spontaneous VT in patients did not show acceleration of VT cycle length or degeneration into VF the introduction of one or multiple ventricular extrasystoles may be arrhythmogenic in individual patients or during prolonged stimulation periods. In such patients a modification of the device overcomes this important limitation by delivering ventricular extrastimuli from specific ventricular sites from which it is less likely to induce or accelerate ventricular arrhythmias. For this purpose, the coupled stimuli will be introduced via a ventricular lead which is positioned either close to or at the His-bundle (His- or para-His site) or via a lead system that allows simultaneous right and left ventricular pacing. As both these stimulation modes enable the coupled beat to excite the ventricles with narrow QRS complexes either by using the specialized natural His-Purkinje conduction system or by fusing left and right ventricular excitation the arrhythmogeneity of the paced beats is minimized.

Although the first spontaneous beat after S-1 is a VT-beat with augmented contractility due to the postextrasystolic potentiation this VT-beat is still characterized by a dyssynchronous ventricular contraction pattern due to the broad QRS complex. To further augment ventricular contraction force, a specific embodiment of the device will also replace each successive spontaneous VT beat occurring after the coupled beat (S-1) by a stimulated beat from the (para-) His site or from a biventricular stimulation site. To achieve this, another paced beat S-2 is introduced after each coupled beat S-1 so that S-2 excites the ventricles briefly before the anticipated next spontaneous VT beat. The second coupling interval of S-2 (CI-2) is either calculated by the device (VT-cycle length×2−coupling interval of S-1) or empirically measured during short periods of spontaneous VT with only S-1 introduction (CI=interval between S-1and earliest ventricular activation by subsequent VT beat−x ms with x ranging from 0-100 ms). S-2 may coincide with the earliest onset of ventricular activation by the spontaneous VT beat or precede/follow this earliest activation by a predefined value which lies typically between 1 and 100 ms. By replacing or fusing with the spontaneous VT beat the ventricles will be depolarized more synchronously than during a VT beat (either via the specific conduction system or via right-/left-/biventricular stimulated beats) which results in a stronger and more efficient depolarization of the beat. Specifically during VT originating from the right (left) ventricle S-2 from the left (right) ventricle triggered onto the spontaneous VT beat after S-1 will narrow the QRS beat of this spontaneous VT beat and increase its hemodynamic efficacy.

During S-2 the device, i.e the control device 1.1, will automatically measure the width of the ventricular electrogram and compare it to the length of the ventricular electrogram of the spontaneous VT beat. The CI-2 is then shortened with each consecutive spontaneous VT beat until the width of the ventricular electrogram approaches (constant) shortest values. The device will take the CI-2 with the shortest ventricular depolarization time (electrogram width) as preferred CI-2. As S-2 may in specific circumstances advance ventricular depolarization (as compared to the spontaneous VT beat) this may shorten the diastolic interval between S-1 and S-2. In those instances in which the CI-2 is shorter than the interval between S-1 and the earliest activation of the successive spontaneous VT beat the benefit of a synchronized (narrow QRS) S-2 beat may partially outweigh the prolonged filling time. Therefore, the device chooses a CI-2 which is defined by 2 concurring criteria:

1) CI-2 will not be shorter than x% of the interval between S-1 and earliest activation by the next spontaneous VT beat.

2) The ventricular electrogram width (QRS width) caused by S-2 will have to be y% shorter than the electrogram width during a spontaneous VT beat. X and y are operator/physician based values and can be programmed to the device as needed.

To facilitate programming, the device provides a normogramm which adjusts the CI-2 to the VT cycle length and individual hemodynamic condition of the patient (e.g. ejection fraction). The normogramm is established either on an empiric way or by hemodynamic testing during or after the implantation procedure. Alternatively, a hemodynamic flow-sensor or pressure sensor incorporated or attached to the device may be used to optimize the CI-2.

In another embodiment of the invention, in a continuous replacement stimulation mode, the device 1 will accelerate the pulse wave rate during VT by continuous atrial or ventricular replacement stimulation (CRS) at a cycle length slightly shorter than the spontaneous VT cycle length (series of S-3 stimuli). Pacing stimuli are either delivered to the atria via an atrial lead or are delivered to the His bundle or atrial/ventricular insertion of the His bundle via a lead close to or at the His bundle. Alternatively, stimuli may be delivered through a ventricular pacing lead fixed at a right ventricular septal or pulmonary outflow tract site. Stimuli can also be delivered via endo- or epicardial (left-) biventricular pacing leads. Thereby, the ventricles will be excited at a slightly faster rate than during spontaneous VT but in a more synchronous fashion than during VT which will result in an increase of arterial blood pressure.

The magnitude of the benefit associated with an improved ventricular contraction pattern at shorter cycle length during CRS from atrial, Para-His, His-, RVOT-ventricular septal or at biventricular stimulation sites as compared to the contraction pattern during spontaneous VT at slightly longer VT-CL critically depends on the third coupling interval of the replacement stimuli (CI-3). Therefore, during VT CRS-stimuli will be introduced with an initial coupling interval CI-3 equaling VT-cycle length−z ms (z ranging from 0-100 ms, typically being 10 ms). The width of the ventricular electrogram will be automatically measured and compared to the width during spontaneous VT. During successive beats the coupling interval will be decreased stepwise (step size 1-10 ms) until a minimal coupling interval is reached (defined as VT CL-zmax) or until the electrogram width approaches a constant minimal value or a value which is close to the width during normal sinus rhythm (or AF). Z and zmax are operator/physician based values and can be programmed to the device as needed. To facilitate programming, the device provides a normogramm which adjusts the CI-3 to the VT cycle length and individual hemodynamic condition of the patient (e.g. ejection fraction). The normogram is established either on an empiric way or by hemodynamic testing during or after the implantation procedure. Alternatively, a hemodynamic flowsensor or pressure sensor incorporated or attached to the device may be used to optimize the CI-3.

In an alternative embodiment, the device 1 is connected to least 2 ventricular pacing/sensing leads, one of said ventricular pacing/sensing leads being connected to the second interface 1.3, the other one of said ventricular pacing/sensing leads being connected to the fourth interface 1.5.

While one of the leads is localized in/on the left ventricle the second one is positioned in/on the right ventricle. Depending on the origin of a spontaneous VT within the left or right ventricle, ventricular activation will be detected earlier in the left or right ventricular leads.

In order to allow for a more synchronized ventricular contraction pattern and hemodynamic improvement during VT, in a triggered ventricular stimulation mode, the device 1 delivers triggered ventricular stimuli to the heart over the ventricular electrode which is activated latest during spontaneous VT (triggered ventricular stimulation: TVS). E.g. if ventricular activation during spontaneous VT is earlier in the right (left) ventricular lead than in the left (right) ventricular lead triggered stimuli will be delivered to the heart via the left (right) ventricular lead. The cycle length of the triggered beats during VT is typically equal or slightly longer than the spontaneous VT cycle length but can be programmed to precede spontaneous depolarization if hemodynamically advantageous. In a typical condition the triggered beat will be delivered to the contra-lateral ventricular chamber at the time of earliest ventricular depolarization registered via the lead in the ventricle from which the VT origins.

This will allow for a simultaneous contraction of the right and left ventricle during VT thereby functionally rendering a VT to a SVT while during spontaneous VT the left (right) ventricle and especially the lateral wall of the left (right) ventricle contracts after the right (left) ventricle and after the interventricular septal wall which results in a dyssynchronous contraction of the ventricles.

Further modifications of the device specifically deliver TVS with PVS to combine 2 beneficial effects for augmentation of contractile force of the heart. In such scenario, the paired stimulus will prevent a breakthrough of very 2nd (xth) VT beat and cause a post-extrasystolic potentiation of the succeeding VT beat which in turn is additionally augmented by TVS.

The device 1 may also be used to slow the arterial pulse wave rate by delivering paired stimuli during atrial fibrillation with rapid atrioventricular nodal conduction as suggested in Yamada H et al. Am J Physiol Heart Circ Physiol. 2003; 285: H2630-8.

If the ventricular cycle length during AF decreases below a predefined interval a ventricular extra-beat is initiated with a coupling interval just outside the ventricular refractory period. This VPB with short coupling interval does not produce any or a sufficient pressure wave as the diastolic filling time of the heart is too short. By the same time, the VPB attenuates the conduction of fibrillating atrial excitations over the AV node by retrograde penetration of the VPB into the AV node. Moreover, the VPB resets/prolongs the ventricular refractory period. Therefore any excitation which antegradely penetrates the AV node will not be able to depolarize the ventricles until after the refractory period of the VPB. By prolonging diastolic filling time and postextrasystolic potentiation the next atrial excitation conducted to the ventricles via the AV node elicits an augmented ventricular contraction with increased contractile force of the ventricles. The major disadvantage of an approach described in Yamada H et al. Am J Physiol Heart Circ Physiol. 2003; 285: H2630-8, however, lies in the fact that by delivering very early ventricular premature beats during tachycardic AF, ventricular tachyarrhythmias may be induced especially in otherwise diseased hearts. The present invention solves this problem by delivering coupled ventricular premature beats during AF via a stimulation lead positioned at Para-His, His-, ventricular septal or RVOT- or at biventricular stimulation sites. As premature ventricular depolarization via the natural ventricular conduction (His-Purkinje) system or with narrow QRS complexes (biventricular pacing) is less arrhythmogenic paired stimulation during AF with rapid ventricular response will reduce the ventricular rate during AF while preventing the induction of VT or VF.

As the asynchronous ventricular contraction pattern during supraventricular tachycardia (SVT) with (functional) bundle branch block (BBB) causes hemodynamic deterioration similar to a VT, PVS or TVS will be also delivered by the device in these cases.

For this purpose, a modification of the device 1 will be connected via a fifth interface 1.6 to an atrial sensing electrode and a electrogram algorithm for diagnosis of rate dependant bundle branch block. If the atrial deflection precedes the ventricular activation in a 1:1 fashion by a predefined time interval or if atrial fibrillation is detected and the ventricular lead simultaneously senses QRS complexes longer than 120 ms a compare algorithm will be initiated: This compare algorithm is based on intracardiac electrocardiogram morphology templates which have been gathered during device programming: during such programming, atrial rapid pacing at various frequencies between 100 and 240 beats/min. will be performed and intracardiac ventricular signals will be recorded. In parallel, 12-lead surface ECG will be recorded to verify at which frequency bundle branch block occurs and to align a specific ventricular intracardiac QRS width and morphology with the surface ECG diagnosis of rate dependant bundle branch block. These templates will then be stored in the defibrillator or pacemaker and allow for a specific differential diagnosis of rate dependant bundle branch block during SVT vs. VT.

If supraventricular tachycardia with functional bundle branch block arises and has been identified by the device 1, the device will deliver ventricular paired stimuli to augment LV contractility. Alternatively the device will deliver triggered stimuli to the chamber, which is excited later (e.g. left ventricle during LBBB, right ventricle during RBBB) with the triggered stimuli being delivered onto the sensed ventricular event as described above. Also, premature atrial/ventricular paired stimuli will be delivered to the atrial/ventricular tachycardic beats to prevent antegrade or retrograde penetration of every 2nd or xth atrial/ventricular into the AV node during tachycardia. Besides promoting concealed conduction and intermittent blockade of the AV nodal conduction capabilities these atrial/ventricular premature beats will cause atrial/ventricular postextrasystolic augmentation which will further contribute to an augmentation of left ventricular contractile force.

If the device 1 is used for hemodynamic stabilization during ventricular tachycardia different adjustments to competing ICD based therapies of VT are incorporated into the device:

If the ICD is not able to terminate VT after a preprogrammed time interval or after a set number of ATP or cardioversion therapies the PVS therapy will be initiated to hemodynamically stabilize the patient. This is the typical situation in a patient with recurrent or incessant VT. At the same time, a signal (e.g. acoustic signal) is sent to the patient to inform him that immediate contact with the emergency service or physician is necessary to initiate e.g. additional antiarrhythmic drug therapy. At the same time or alternatively the emergency system is automatically informed by the device via a telemetric signal of the identity and localization of the patient (e.g. via GPS). This may be achieved by transmitting signals to a patient's mobile phone or wearable or integrated transmission box.

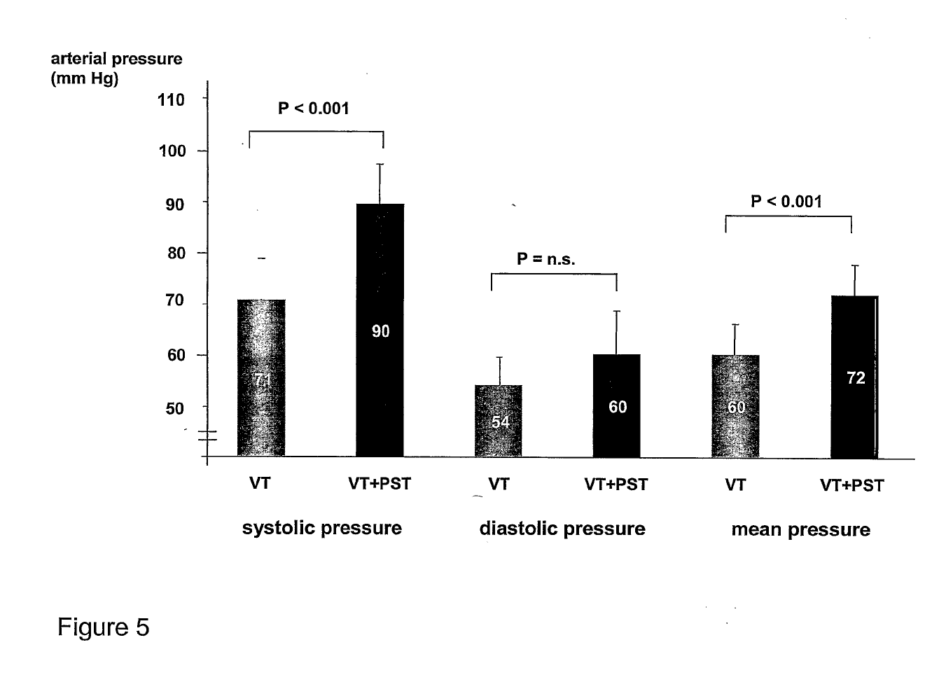

Also, the device may allow for a hemodynamic stabilization during anti-tachycardic (overdrive) ventricular pacing attempts (ATP) to terminate VT. Currently, the number of ATP attempts is limited as the tachycardia itself and the further increase of the ventricular rate during ATP may deteriorate cardiac output. Consequently, a cardioversion shock is usually initiated after a predefined time interval. The device solves this dilemma by intermittently introducing short episodes of PVS and/or TVS to allow for short-time hemodynamic recovery after which repeated ATP attempts, which then terminate the arrhythmia without CV, are delivered. A representative example is illustrated in FIG. 5.

The duration of such combined ATP/PVS/TVS attempts depends on the tachycardia cycle length and patient condition and is predefined by the physician. If a hemodynamic sensor is incorporated into the device the duration of PVS/TVS can be automatically adjusted to the hemodynamic condition o the patient. In such case the cardiac output values during VT and PVS/TVS are compared to those during SR. If the integral of cardiac output/arterial pressure over a time interval is below a predefined value the ATP/PVS/TVS attempts are terminated an a CV shock is initiated.

Finally, in cases in which ATP fails a CV shock will be delivered by the ICD. To prevent a hemodynamic collapse during charging of the shock voltage onto the capacitors PVS/TVS will be delivered during ICD charging. This will allow to prevent syncope of the patient before the shock delivery thereby avoiding possible accompanying physical damage to the patient during syncope (e.g. traffic accident).

1. Implantable device, in particular implantable cardioverter-defibrillator, for responding to tachycardia events in a patient's heart comprising
a control device,
a first interface connected to said control device for receiving first signals representative of tachycardia events and connectable to a first sensor device for detecting tachycardia events,
a second interface connected to said control device and connectable to a first stimulation electrode, and
said control device being arranged for providing at least one stimulation pulse to said second interface in response to at least one of said first signals received at said second interface for responding to tachycardia events
wherein
said control device is arranged for providing at least one first stimulation pulse to said second interface upon continued presence of said first signals at said first interface for at least intermittently improving the cardiac output during continued tachycardia events.

2. Implantable device according to claim 1, wherein
said control device has a paired ventricular stimulation (PVS) mode wherein said control device provides said first stimulation pulse to said second interface in response to each n-th first signal at termination of a predetermined first coupling interval, said first coupling interval being adapted to substantially suppress a spontaneous second tachycardia event immediately following a first tachycardia event causing said n-th first signal,
said control device is switched to said paired ventricular stimulation (PVS) mode during a predetermined first operation interval in response to continued presence of said first signals at said first interface.

3. Implantable device according to claim 2, wherein n is an integer greater than 1.

4. Implantable device according to claim 2 or 3, wherein said first coupling interval is adapted to end shortly after the termination of the ventricular refractory period following said first tachycardia event.

5. Implantable device according to any one of claims 2 to 4, wherein said control device is adapted to provide at least a second stimulation pulse to said second interface, said second stimulation pulse following said first stimulation pulse at termination of a predetermined second coupling interval, said second coupling interval being adapted to substantially replace or fuse with a spontaneous third tachycardia event immediately following said suppressed spontaneous second tachycardia event.

6. Implantable device according to claim 5, wherein said second coupling interval is adapted to end shortly before an estimated occurrence of said third tachycardia event.

7. Implantable device according to claim 5 or 6, wherein
said control device is adapted to receive second signals representative of the ventricular electrogram of said patient's heart,
said control device is adapted to evaluate the width of a first ventricular electrogram received after provision of a second stimulation pulse, and
said control device is adapted to modify the duration of said second coupling interval, in particular to reduce the duration of said second coupling interval, until a minimum width of said first ventricular electrogram is reached.

8. Implantable device according to any one of the preceding claims wherein
said control device has a continuous replacement stimulation (CRS) mode wherein said control device provides said first stimulation pulse to said second interface at a first cycle length slightly shorter than the spontaneous tachycardia event cycle length between subsequent spontaneous tachycardia events,
said control device is switched to said continuous replacement stimulation (CRS) mode during a predetermined second operation interval in response to continued presence of said first signals at said first interface.

9. Implantable device according to claim 8, wherein said control device is adapted to provide said first stimulation pulse to said second interface at termination of a predetermined third coupling interval, said third coupling interval being adapted to substantially replace a spontaneous tachycardia event immediately following a preceding first stimulation pulse.

10. Implantable device according to claim 8 or 9, wherein said third coupling interval is slightly shorter than said spontaneous tachycardia event cycle length.

11. Implantable device according to claim 9 or 10, wherein
said control device is adapted to receive second signals representative of the ventricular electrogram of said patient's heart,
said control device is adapted to evaluate the width of a first ventricular electrogram received after provision of a first stimulation pulse, and
said control device is adapted to modify the duration of said third coupling interval, in particular to reduce the duration of said third coupling interval, until
a minimum width of said first ventricular electrogram is reached or
a predetermined minimum value of said third coupling interval is reached or
a width of said first ventricular electrogram which substantially corresponds to a normal width of said first ventricular electrogram during normal sinus rhythm.

12. Implantable device according to any one of the preceding claims, wherein
said first interface incorporates said second interface,
said control device has a first sensing mode and is adapted to receive said first signals from said first interface in said first sensing mode, and
said control device has a first pacing mode and is adapted to provide said first stimulation pulse to said first interface in said first pacing mode.

13. Implantable device according to any one of the preceding claims further comprising
a third interface connected to said control device for receiving second signals representative of tachycardia events and connectable to a second sensor device for detecting tachycardia events,
a fourth interface connected to said control device and connectable to a second stimulation electrode, and
said control device being arranged for providing at least one stimulation pulse to said fourth interface in response to at least one of said second signals received at said third interface for responding to tachycardia events,
said control device having a triggered ventricular stimulation (TVS) mode wherein
said control device provides said first stimulation pulse to said second interface if one of said second signals is received at said third interface prior to receiving a first signal at said first interface and
said control device provides said second stimulation pulse to said fourth interface if one of said first signals is received at said first interface prior to receiving a second signal at said third interface, and
said control device being switched to said triggered ventricular stimulation (TVS) mode during a predetermined third operation interval in response to continued presence of said first signals at said first interface.

14. Implantable device according to claim 13, wherein
said third interface incorporates said fourth interface and
said control device has a second sensing mode and is adapted to receive said second signals from said third interface in said second sensing mode, and
said control device has a second pacing mode and is adapted to provide said second stimulation pulse to said third interface in said second pacing mode.

15. Implantable device according to any one of the preceding claims further comprising
a fifth interface connected to said control device for receiving third signals representative of atrial tachycardia events and connectable to a third sensor device for detecting atrial tachycardia events,
a first memory connected to said control device, said first memory storing an electrogram algorithm and a plurality of previously established electrogram templates,
said control device having a discrimination mode wherein said control device, using said electrogram algorithm and at least a part of said plurality of previously established electrogram templates, determines from said third signals and at least said first signals if a supraventricular tachycardia with functional bundle branch block prevails
said control device having a supraventricular tachycardia (SVT) treatment mode for treating supraventricular tachycardia (SVT), and
said control device being switched to said supraventricular tachycardia (SVT) treatment mode if a supraventricular tachycardia is determined in said discrimination mode.

16. Implantable device according to any one of the preceding claims, wherein
said control device has an antitachycardic pacing (ATP) mode for terminating tachycardia events by providing antitachycardic pacing stimulation pulses to at least said second interface,
and/or
said control device has an cadioversion (CV) shock mode for terminating tachycardia events by providing cadioversion (CV) shock stimulation pulses to at least said second interface.
